(19) United States
(12) Patent Application Publication
(10) Pub. No.: US 2018/0078304 A1
(43) Pub. Date: Mar. 22, 2018

(54) DEVICE FOR EVACUATING SMOKE DURING SURGICAL PROCEDURES

(71) Applicant: Eesha Rushmie Bhattacharyya, Kailua, HI (US)

(72) Inventor: Eesha Rushmie Bhattacharyya, Kailua, HI (US)

(21) Appl. No.: 15/698,352

(22) Filed: Sep. 7, 2017

Related U.S. Application Data
(60) Provisional application No. 62/395,905, filed on Sep. 16, 2016.

(51) Int. Cl. A61B 18/14

(52) U.S. Cl. CPC A61B 18/1482; A61B 2217/005; A61B 2218/008

(57) ABSTRACT

A device for evacuating smoke during a surgical procedure may include a tube configured to engage with a surgical instrument; a stopcock extending from the tube; and a suction tubing attached to the stopcock, such that when the surgical instrument is being used and smoke is being produced the smoke can be evacuated through the tube and stopcock. The tube may be a tube with external claspers designed to engage with the surgical instrument or a hollow tube with a diameter sufficient to accommodate the surgical instrument therein, wherein the smoke is evacuated around the surgical instrument.

### RELATED APPLICATION

This application claims priority to provisional patent application U.S. Ser. No. 62/395,905 filed on Sep. 16, 2016, the entire contents of which is herein incorporated by reference.

### BACKGROUND

The embodiments herein relate generally to medical devices and, more particularly, to a device for evacuating smoke during surgical procedures and, specifically, during laparoscopic procedures.

Smoke generated from laparoscopic electrosurgery or harmonic desiccation can obscure the surgical visual field. Currently, suctioning and irrigation of the surgical field is done through a separate port. Alternatively, the surgeon simply has to wait for the smoke to clear the area.

Therefore, what is needed is a device for evacuating smoke during a surgical procedure, wherein the suction is allowed to occur at the site of the smoke production through the same port as the surgical instrument.

### SUMMARY

Some embodiments of the present disclosure include a device for evacuating smoke during a surgical procedure. The device may include a tube configured to engage with a surgical instrument; a stopcock extending from the tube; and a suction tubing attached to the stopcock, such that when the surgical instrument is being used and smoke is being produced the smoke can be evacuated through the tube and stopcock. The tube may be a tube with external claspers designed to engage with the surgical instrument or a hollow tube with a diameter sufficient to accommodate the surgical instrument therein, wherein the smoke is evacuated around the surgical instrument.

### BRIEF DESCRIPTION OF THE FIGURES

The detailed description of some embodiments of the invention is made below with reference to the accompanying figures, wherein like numerals represent corresponding parts of the figures.

FIG. 1 is a perspective view of one embodiment of the present disclosure.

FIG. 2 is a perspective view of one embodiment of the present disclosure.

FIG. 3 is a perspective view of one embodiment of the present disclosure.

FIG. 4 is an exploded view of one embodiment of the present disclosure.

FIG. 5 is a perspective view of one embodiment of the present disclosure.

FIG. 6 is a perspective view of one embodiment of the present disclosure.

FIG. 7 is a perspective view of one embodiment of the present disclosure.

FIG. 8 is a perspective view of one embodiment of the present disclosure.

FIG. 9 is a perspective view of one embodiment of the present disclosure.

### DETAILED DESCRIPTION OF CERTAIN EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

The device of the present disclosure may be used to evacuate smoke during a laparoscopic surgery and may comprise the following elements. This list of possible constituent elements is intended to be exemplary only, and it is not intended that this list be used to limit the device of the present application to just these elements. Persons having ordinary skill in the art relevant to the present disclosure may understand there to be equivalent elements that may be substituted within the present disclosure without changing the essential function or operation of the device.

a. Tube

b. Stopcock

c. Suction Tubing

The various elements of the device of the present disclosure may be related in the following exemplary fashion. It is not intended to limit the scope or nature of the relationships between the various elements and the following examples are presented as illustrative examples only.

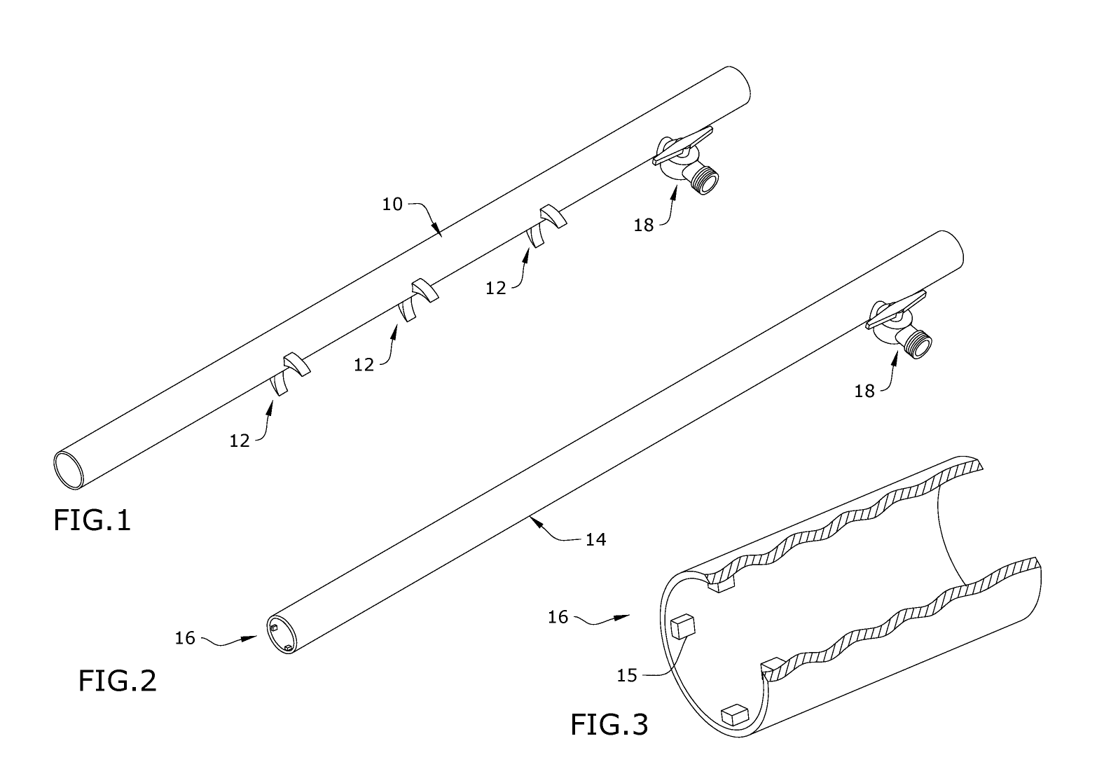

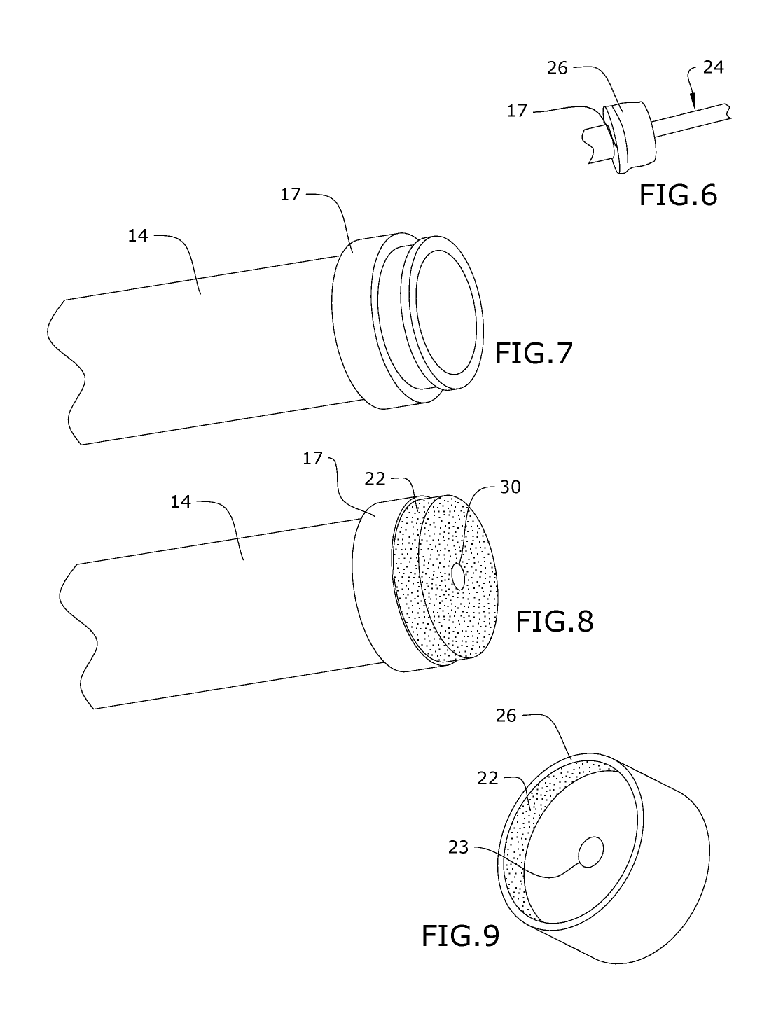

By way of example, and referring to FIGS. 1-9, some embodiments of the present disclosure include a device for evacuating smoke during a surgical procedure, the device comprising a tube designed to engage with a surgical instrument, a stock cock 18 extending from the tube, and a suction tubing (not shown) attached to the stopcock 18, such that when the surgical instrument is being used and smoke is being produced, a user may evacuate the smoke using the stopcock 18.

In some embodiments, such as that shown in FIG. 1, the tube may comprise a substantially cylindrical tube 10 with a plurality of claspers 12 attached to an outer surface of the cylindrical tube 10, the claspers 12 being designed to removably engage with the surgical instrument. In such embodiments, a first end of the tube 10 may be open, wherein the open end is positioned proximate to the surgical area during use, and a second end of the tube 10 may be open or closed.

Alternatively, and as shown, for example, FIGS. 2-9, the tube may comprise a substantially cylindrical tube 14, such as a larger bore circular tube, wherein the tube 14 has a diameter sufficient to accommodate the surgical instrument therein. As shown in FIGS. 2 and 3, a first end 16 of the tube 14 may include a plurality of protruding guides 15 extending from an inner surface thereof towards a center area thereof, wherein the protruding guides 15 may function to stabilize the surgical instrument within the tube 14. Additionally, a cap may be designed to engage with the end of the tube 14 proximate to the stopcock 18 and distal from the protruding guides 15, wherein the cap includes a cap orifice 23 extending therethrough, such that a shaft 24 of a surgical instrument may extend through the cap, into the tube 14, and out of the distal end 16 of the tube 14, wherein the protruding guides 15 may assist in stabilizing the surgical tool and ensuring that there is space between the surgical tool and an inner surface of the tube 14 such that smoke may be evacuated around the tool while the tool is in use. In the case of the tube 14, a diameter of the tube 14 may be larger than a diameter of a shaft 24 of the surgical tool to be used.

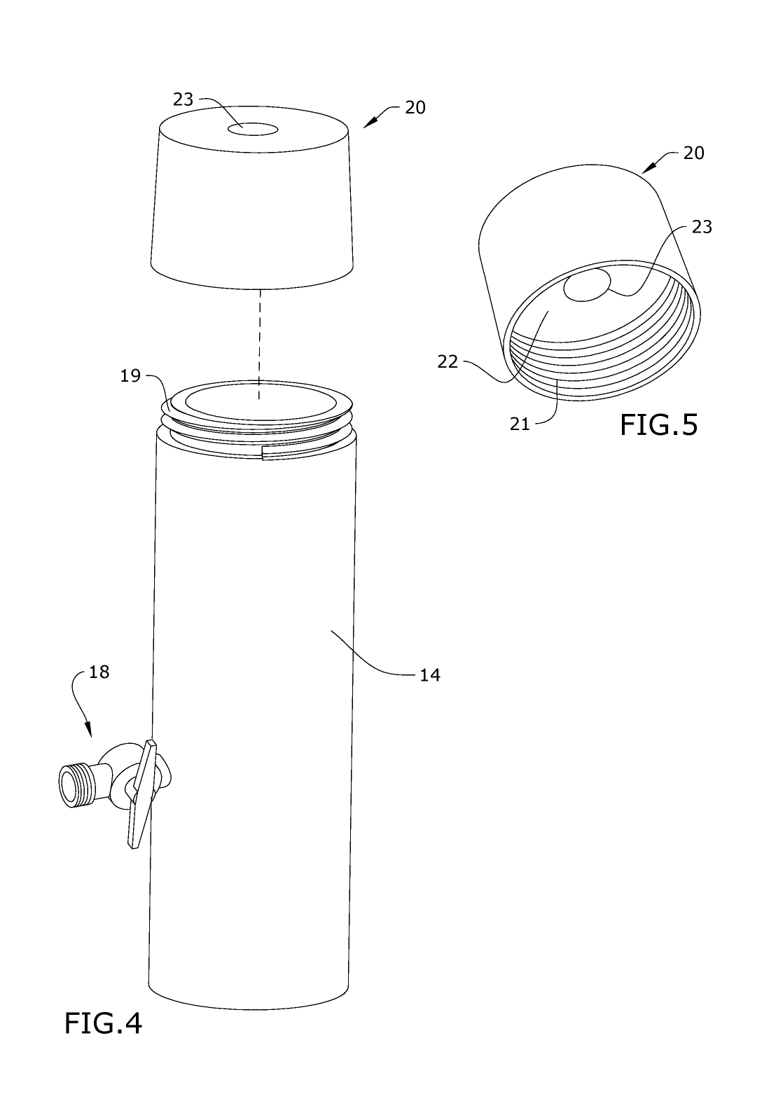

In some embodiments, such as those shown in FIGS. 4 and 5, an end of the tube proximate to the stopcock 18 and distal from the protruding guides 15 may comprise tube threads 19. A cap 20 designed to engage with the tube 14 may comprise cap threads 24 on an inner surface thereof, such that the cap 20 may screw onto the tube 14.

In other embodiments, such as those shown in FIGS. 6-9, the tube 14 may comprise a cylindrical neck sleeve 17 positioned around an end of the tube 14 distal from the protruding guides 15, wherein the cylindrical neck sleeve 17 is spaced from the end by a predetermined distance. A gasket 22 may be positioned around the end of the tube 14, wherein the gasket comprises a gasket orifice 30 in an end thereof. A rubberized cap 26 may then be positioned over the gasket 22, such that the rubberized cap 26 abuts the cylindrical neck sleeve 17 and a cap orifice 23 in the rubberized cap 26 aligns with the gasket orifice 30.

Alternatively, the cap may engage with the tube using any known connection means. For example, the cap may snap into place. However, in any case, the cap orifice 23 may be positioned such that the shaft 24 of the tool may extend through the cap and to the distal end 16 of the tube 14. For example, in embodiments, the cap orifice 23 may be concentrically aligned with the cap and the tube 14.

In some embodiments, the surgical tool or instrument may comprise an electrosurgical or harmonic instrument, such as a laparoscopic instrument.

To use the device of the present disclosure, a user may engage the tube with the surgical instrument, attach a suction tube to the stopcock 18, and perform suction as normal. When smoke needs to be evacuated, the user may use the stopcock 18 to control the suction power, resulting in the smoke being evacuated through the tube. Moreover, in embodiments, an irrigation line may additionally be attached to the tube, such that smoke evacuation and irrigation of bleeding areas to improve visualization may each be performed without having to remove and reinsert tools during a procedure.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A device for evacuating smoke during a surgical procedure, the device comprising:
a tube configured to engage with a surgical instrument;
a stopcock extending from the tube; and
a suction tubing attached to the stopcock, such that when the surgical instrument is being used and smoke is being produced the smoke can be evacuated through the tube and stopcock.

2. The device of claim 1, wherein the tube is a member selected from the group consisting of a tubing with external claspers configured to engage with the surgical instrument and a hollow tube with a diameter sufficient for accommodating the surgical instrument.

3. The device of claim 2, wherein the tube is the tubing with external claspers, the tubing with external claspers comprising:
a cylindrical tubing; and
a plurality of claspers extending from the cylindrical tubing, the plurality of claspers being spaced along a length of the cylindrical tubing.

4. The device of claim 2, wherein the tube is a hollow tube with a diameter sufficient for accommodating the surgical instrument, the hollow tube comprising:
a hollow tube; and
a cap attached to the end of the hollow tube, the cap comprising a cap orifice extending therethrough.

5. The device of claim 4, wherein the cap is removable.

6. The device of claim 5, wherein:
an end of the hollow tube designed to engage with the cap comprises tube threads; and
an interior surface of the cap comprises cap threads designed to engage with the tube threads.

7. The device of claim 5, wherein:
a cylindrical neck sleeve is positioned around an end of the hollow tube designed to engage with the cap;
a gasket is positioned adjacent to the cylindrical neck sleeve, wherein the gasket comprises a gasket orifice extending therethrough; and
the cap comprises a rubberized cap with designed to surround the gasket, wherein the cap orifice is positioned to align with the gasket orifice.

8. The device of claim 4, wherein:
a plurality of guide protrusions extend from an inner surface of an end of the hollow tube distal from the cap; and
the plurality of guide protrusions are sized to center the surgical instrument.
